(19) United States
(12) Patent Application Publication
(10) Pub. No.: US 2026/0007376 A1
(43) Pub. Date: Jan. 8, 2026

(54) X-RAY CT APPARATUS, METHOD OF CONTROLLING X-RAY CT APPARATUS, AND STORAGE MEDIUM

(71) Applicant: CANON MEDICAL SYSTEMS CORPORATION, Otawara-shi (JP)

(72) Inventor: Takahiro YODA, Nasushiobara (JP)

(73) Assignee: CANON MEDICAL SYSTEMS CORPORATION, Otawara-shi (JP)

(21) Appl. No.: 19/254,153

(22) Filed: Jun. 30, 2025

(30) Foreign Application Priority Data
Jul. 5, 2024 (JP) 2024-108870

(51) Int. Cl. A61B 6/42; A61B 6/00; A61B 6/03; A61B 6/46

(52) U.S. Cl. CPC A61B 6/4241; A61B 6/032; A61B 6/463; A61B 6/481; A61B 6/5205

(57) ABSTRACT

An X-ray CT apparatus includes processing circuitry. The processing circuitry acquires at least first count data and second count data having different energy bands as count data of X-ray photons detected by an X-ray detector, generates adjusted count data based on relative weighting factors between the first count data and the second count data, and reconstructs the adjusted count data.

### CROSS-REFERENCE TO RELATED APPLICATION

The present application claims priority based on Japanese Patent Application No. 2024-108870 filed Jul. 5, 2024, the content of which is incorporated herein by reference.

### FIELD

Embodiments disclosed in the present specification and drawings relate to an X-ray CT apparatus, a method of controlling the X-ray CT apparatus, and a storage medium.

### BACKGROUND

A photon counting computed tomography (CT) apparatus is an apparatus that detects each photon with an X-ray detector and counts detected photons to measure X-rays. In a photon counting CT apparatus, an X-ray detector discriminates and collects photons for each energy band with respect to energy bins (hereinafter sometimes abbreviated as bins). The photon counting CT apparatus generates projection data based on count data indicating the number of X-ray photon counts for each bin, reconstructs the projection data, and generates reconstructed image data for displaying a reconstructed image.

A photon counting CT apparatus generates reconstructed image data by reconstructing the projection data using spectral reconstruction, for example. Alternatively, a photon counting CT apparatus reconstructs the projection data by integrating outputs of the bins in the X-ray detector and reconstructing the integration result as a single energy, or by processing an output of a specific bin as a single energy.

In recent years, in some examination using a photon counting CT apparatus or the like, there has been a demand for functions such as fluoroscopy and RealPrep, which allow images to be observed in real time. However, the technique for reconstructing the projection data based on spectral reconstruction has some limitations on a processing speed, and therefore it has been impossible to apply this technique to a technique of observing images in real time.

### BRIEF DESCRIPTION OF THE DRAWINGS

FIG. 1 is a diagram showing an example of an X-ray CT apparatus 1 according to an embodiment.

FIG. 2 is a diagram showing an example of a configuration of a DAS 16 according to the embodiment.

FIG. 3 is a diagram showing an example of functional blocks for a reconstruction function 53 according to the embodiment.

FIG. 4 is a diagram showing an example of functional blocks for an adjustment function 56 according to the embodiment.

FIG. 5 is a flowchart showing an example of processing in the X-ray CT apparatus 1.

FIG. 6 is a diagram visualizing change in the ratio of weighting factors each time one view is executed.

FIG. 7 is a diagram for explaining an example of a process for generating a reconstructed image.

FIG. 8 is a diagram showing an example of a control device 18 of a third embodiment.

### DETAILED DESCRIPTION

An X-ray CT apparatus, a method of controlling the X-ray CT apparatus, and a program according to embodiments will be described below with reference to the drawings.

An X-ray CT apparatus of an embodiment includes processing circuitry that acquires at least first count data and second count data having different energy bands as count data of X-ray photons detected by an X-ray detector, generates adjusted count data based on relative weighting factors between the first count data and the second count data, and reconstructs the adjusted count data.

The X-ray CT apparatus of the embodiment is a photon counting CT apparatus. A photon counting CT apparatus discriminates materials through which X-rays have passed using a direct-type detector. In the X-ray CT apparatus of the embodiment, when a reconstructed image is generated, weighted count data is used to generate projection data and reconstructed image data, to reduce the amount of calculation processing, thereby shortening the time required to generate a reconstructed image.

### First Embodiment

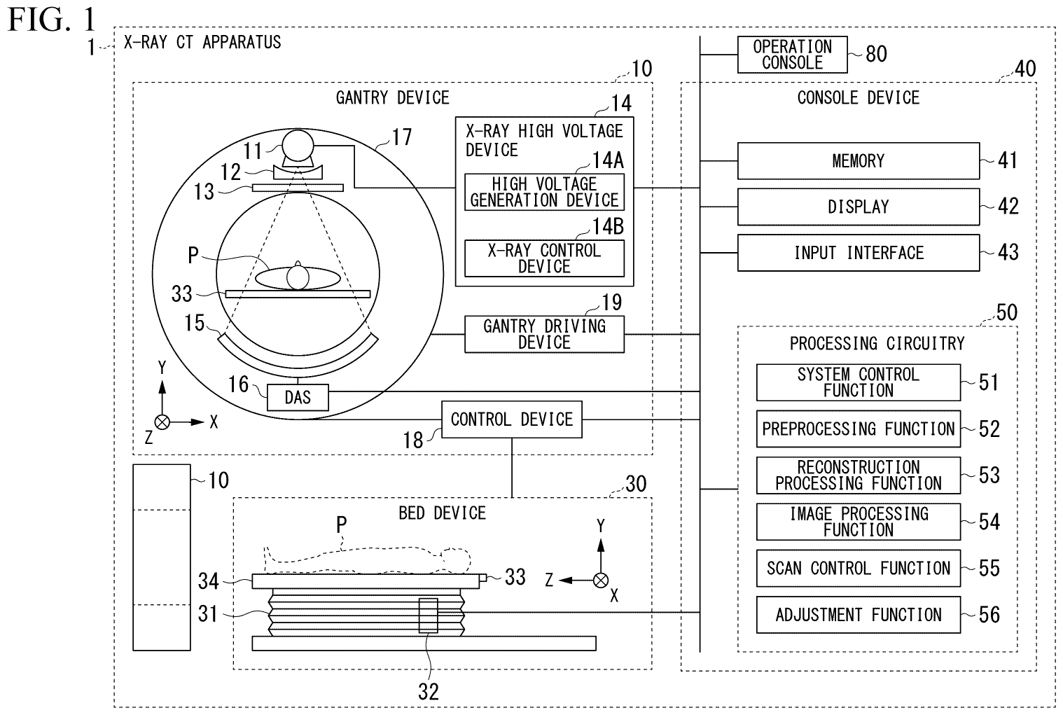

FIG. 1 is a diagram showing an example of an X-ray CT apparatus 1 according to a first embodiment. The X-ray CT apparatus 1 includes, for example, a gantry device 10, a bed device 30, a console device 40, and an operation console 80. Of these components, the gantry device 10, the bed device 30, and the operation console 80 are installed, for example, in an examination room, and the console device 40 is provided outside the gantry device 10 and installed in an operation room adjacent to the examination room. The console device 40 may be installed in the examination room, and the operation console 80 may be installed in the operation room.

For convenience of description, FIG. 1 shows both of an illustration of the gantry device 10 viewed in the Z-axis direction and an illustration of the same viewed in the X-axis direction, but in reality there is only one gantry device 10. In the present embodiment, a rotation axis of a rotating frame 17 in a non-tilted state or a longitudinal direction of the top board 33 of the bed device 30 is defined as the Z-axis direction; an axis orthogonal to the Z-axis direction and horizontal to the floor surface is defined as the X-axis direction, and a direction orthogonal to the Z-axis direction and perpendicular to the floor surface is defined as an Y-axis direction.

The gantry device 10 includes, for example, an X-ray tube 11, a wedge 12, a collimator 13, an X-ray high voltage device 14, an X-ray detector 15, a data acquisition system (hereinafter referred to as DAS) 16, the rotating frame 17, a control device 18, and a gantry driving device 19. The X-ray tube 11, the wedge 12, the collimator 13, the X-ray high voltage device 14, the X-ray detector 15, the DAS 16, the rotating frame 17, and the control device 18 are built into the gantry device 10. The rotating frame 17 rotatably holds the X-ray tube 11 and the X-ray detector 15.

The X-ray tube 11 generates X-rays by radiating thermoelectrons from a cathode (filament) to an anode (target) when a high voltage is applied thereto from the X-ray high voltage device 14. The X-ray tube 11 includes a vacuum tube. For example, the X-ray tube 11 is a rotating anode type X-ray tube that generates X-rays by radiating thermoelectrons to the rotating anode.

The wedge 12 is a filter for adjusting the X-ray dose radiated from the X-ray tube 11 to a subject P. The wedge 12 attenuates the X-rays passing through the wedge 12 so that the distribution of the X-ray dose radiated from the X-ray tube 11 to the subject P becomes a predetermined distribution. The wedge 12 is also called a wedge filter or a bow-tie filter. The wedge 12 is, for example, made of processed aluminum to have a predetermined target angle and a predetermined thickness.

The collimator 13 is a mechanism for narrowing the radiation range of X-rays that have passed through the wedge 12. The collimator 13 narrows the radiation range of X-rays, for example, by forming a slit based on combination of a plurality of lead plates. The collimator 13 is sometimes called an X-ray aperture. The narrowing range of the collimator 13 may be configured to be mechanically drivable.

The X-ray high voltage device 14 includes, for example, a high voltage generation device and an X-ray control device. The high voltage generation device has an electric circuit including a transformer, a rectifier, and so on and generates a high voltage to be applied to the X-ray tube 11. The high voltage generation device may boost the voltage using the above-mentioned transformer or may boost the voltage using an inverter. The X-ray high voltage device 14 may be provided on the rotating frame 17 or on the side of a fixed frame (not shown) of the gantry device 10.

The X-ray detector 15 detects the intensity of X-rays that have been generated by the X-ray tube 11, passed through the subject P and been incident thereon. The X-ray detector 15 outputs an electric signal (alternatively, an optical signal or the like) corresponding to the detected intensity of X-rays to the DAS 16. The X-ray detector 15 has, for example, two or more X-ray detection element rows. Each of the X-ray detection element rows has a number of X-ray detection elements arranged in a channel direction along an arc having the focal point of the X-ray tube 11 as a center thereof. The X-ray detection element rows are arranged in a slice direction (row direction).

The X-ray detector 15 is, for example, a direct detection type detector. For example, a semiconductor diode with electrodes being attached to both ends of a semiconductor is applicable as the X-ray detector 15. X-ray photons incident on the semiconductor are converted into electron-hole pairs. The number of electron-hole pairs generated by incidence of one X-ray photon depends on the energy of the incident X-ray photon. Electrons and holes are attracted to a pair of electrodes formed on both ends of the semiconductor. The pair of electrodes generates electric pulses having peak values according to the charge of electron-hole pairs. One electric pulse has a peak value according to the energy of the incident X-ray photon.

For example, the DAS 16 collects count data indicating the number of counts of X-ray photons detected by the X-ray detector 15 with respect to two or more energy bins in accordance with a control signal from the control device 18. The count data for the energy bins corresponds to an energy spectrum of X-rays incident on the X-ray detector 15, the energy spectrum being modified according to the response characteristics of the X-ray detector 15. The DAS 16 outputs detection data based on a digital signal to the console device 40. The detection data includes digital values of count data identified by a channel number and a row number of an X-ray detection element that is a generation source thereof, and a view number indicating a collected view. The view number is a number that changes according to rotation of the rotating frame 17, which is, for example, a number that is incremented according to rotation of the rotating frame 17. Therefore, the view number is information indicating the rotation angle of the X-ray tube 11. A view period is a period that falls between a rotation angle corresponding to a certain view number and a rotation angle corresponding to the next view number. The DAS 16 may detect switching of a view by a timing signal inputted from the control device 18, by using an internal timer, or by a signal obtained from a sensor that is not shown in the figure. When full scanning is performed and X-rays are continuously emitted by the X-ray tube 11, the DAS 16 collects a group of detection data for the entire circumference (360 degrees). When half scanning is performed and X-rays are continuously emitted by the X-ray tube 11, the DAS 16 collects detection data for half the circumference (180 degrees).

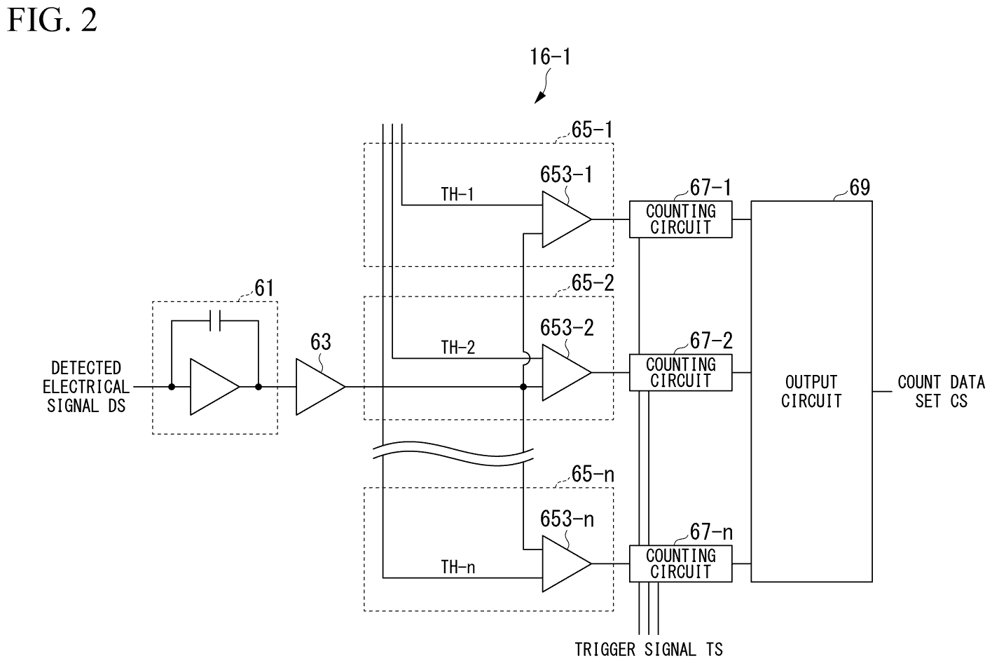

FIG. 2 is a diagram showing an example of a configuration of the DAS 16 according to the embodiment. The DAS 16 has as many readout channels as the number of channels corresponding to the number of X-ray detection elements. These readout channels are implemented in parallel in an integrated circuit such as an application specific integrated circuit (ASIC). FIG. 2 shows only a configuration of a DAS 16-1 that is configured for one readout channel.

The DAS 16-1 includes a preamplifier circuit 61, a waveform shaping circuit 63, a plurality of pulse height discrimination circuits 65, a plurality of counting circuits 67, and an output circuit 69. The preamplifier circuit 61 amplifies a detected electrical signal DS (current signal) from an X-ray detection element that is a connection destination thereof. For example, the preamplifier circuit 61 converts the current signal from the thus-connected X-ray detection element into a voltage signal having a voltage value (peak value) proportional to the charge amount of the current signal. The waveform shaping circuit 63 is connected to the preamplifier circuit 61. The waveform shaping circuit 63 shapes the waveform of the voltage signal from the preamplifier circuit 61. For example, the waveform shaping circuit 63 reduces the pulse width of the voltage signal from the preamplifier circuit 61.

A plurality of counting channels corresponding to the number of energy bands (energy bins) are connected to the waveform shaping circuit 63. When n energy bins are set, n counting channels are provided in the waveform shaping circuit 63. Each counting channel has a pulse height discrimination circuit 65-n and a counting circuit 67-n.

Each pulse height discrimination circuit 65-n discriminates the energy of X-ray photons detected by the X-ray detection element, which is the peak value of the voltage signal from the waveform shaping circuit 63. For example, the pulse height discrimination circuit 65-n has a comparison circuit 653-n. The voltage signal from the waveform shaping circuit 63 is inputted to one input terminal of each comparison circuit 653-n. Reference signals TH (reference voltage values) corresponding to their respective different threshold values are supplied from the control device 18 to the other input terminals of the comparison circuits 653-n, respectively. For example, the comparison circuit 653-1 for an energy bin bin1 is supplied with a reference signal TH-1, the comparison circuit 653-2 for an energy bin bin2 is supplied with a reference signal TH-2, and the comparison circuit 653-n for an energy bin binn is supplied with a reference signal TH-n. Each of the reference signals TH has an upper limit reference value and a lower limit reference value. Each comparison circuit 653-n outputs an electrical pulse signal when the voltage signal from the waveform shaping circuit 63 has a peak value corresponding to the energy bin corresponding to each of the reference signals TH. For example, the comparison circuit 653-1 outputs an electrical pulse signal when the peak value of the voltage signal from the waveform shaping circuit 63 is equal to a peak value corresponding to the energy bin bin1 (when the former peak value is between the reference signals TH-1 and TH-2). On the other hand, the comparison circuit 653-1 for the energy bin bin1 does not output an electrical pulse signal when the peak value of the voltage signal from the waveform shaping circuit 63 is not equal to a peak value corresponding to the energy bin bin1. Further, for example, the comparison circuit 653-2 outputs an electrical pulse signal when the peak value of the voltage signal from the waveform shaping circuit 63 is equal to a peak value corresponding to the energy bin 2 (when the former peak value is between the reference signals TH-2 and TH-3).

The counting circuit 67-n counts electrical pulse signals from the pulse height discrimination circuit 65-n in a readout period that coincides with a switching period of a view. For example, the counting circuit 67-n is supplied with a trigger signal TS from the control device 18 at the switching timing of each view. In the wake of the trigger signal TS having been supplied, the counting circuit 67-n adds 1 to the count number stored in the internal memory each time an electrical pulse signal is input from the pulse height discrimination circuit 65-n. In the wake of the next trigger signal having been supplied, the counting circuit 67-n reads out count number data (i.e., count data) stored in the internal memory and supplies the same to the output circuit 69. Further, the counting circuit 67-n resets the count number stored in the internal memory to an initial value each time the trigger signal TS is supplied. In this way, the counting circuit 67-n counts the count number for each view.

The output circuit 69 is connected to counting circuits 67-n corresponding to a plurality of readout channels mounted on the X-ray detector 15. The output circuit 69 integrates count data from the counting circuits 67-n for the plurality of readout channels for each of the energy bins to generate count data for the readout channels for each view. Count data for each energy bin is a set of count number data defined by a channel, a segment (column), and an energy bin. The count data for each energy bin is transmitted to the console device 40 on a view-by-view basis. Count data for each view is called a count data set CS.

The rotating frame 17 is an annular member by which the X-ray tube 11, the wedge 12, and the collimator 13 are supported together with the X-ray detector 15 in opposition to the X-ray detector 15. The rotating frame 17 is supported by a fixed frame rotatably about the subject P introduced inside the frame 17. The rotating frame 17 also supports the DAS 16. Detection data outputted by the DAS 16 is transmitted through optical communication from a transmitter having a light-emitting diode (LED) provided on the rotating frame 17 to a receiver having a photodiode provided on a non-rotating part (e.g., a fixed frame) of the gantry device 10, and transferred to the console device 40 by the receiver. The method of transmitting the detection data from the rotating frame 17 to the non-rotating part is not limited to the above-mentioned method using optical communication, and any non-contact transmission method may be adopted to this example. The rotating frame 17 is not limited to an annular member and may be an arm-like member as long as it can support and rotate the X-ray tube 11 and others.

The X-ray CT apparatus 1 is, for example, a Rotate/Rotate-Type X-ray CT apparatus (third generation CT) in which both the X-ray tube 11 and the X-ray detector 15 are supported by the rotating frame 17 and rotate around the subject P, but is not limited to this example and may be a Stationary/Rotate-Type X-ray CT apparatus (fourth generation CT) in which a number of X-ray detection elements arranged in an annular shape are fixed to a fixed frame and the X-ray tube 11 rotates around the subject P.

The control device 18 includes processing circuitry having a processor such as a CPU, for example. The control device 18 receives an input signal from the console device 40 or an input interface attached to the gantry device 10 and controls the operations of the gantry device 10, the bed device 30, and the DAS 16. For example, the control device 18 controls the gantry driving device 19 to rotate the rotating frame 17 and/or tilt the gantry device 10. When tilting the gantry device 10, the control device 18 controls the gantry driving device 19 based on an inclination angle (tilt angle) inputted to the input interface to rotate the rotating frame 17 around an axis parallel to the Z-axis direction. The control device 18 recognizes the rotation angle of the rotating frame 17 from the output of a sensor that is not shown, or the like. The control device 18 also provides the rotation angle of the rotating frame 17 to a reconstruction function 53, a scan control function 55, and other functions at any time as needed. The control device 18 also controls the energy bins (reference signals TH) of the DAS 16. The control device 18 may be provided in the gantry device 10 or in the console device 40.

The gantry driving device 19 includes, for example, a motor and an actuator. For example, the gantry driving device 19 rotates the rotating frame 17 and tilts the gantry device 10. The gantry driving device 19 rotates the rotating frame 17 of the gantry device 10 based on an inclination angle (tilt angle) inputted to the input interface and/or a rotation command from a correction data collection function 57 which will be described later.

The bed device 30 is a device that places and moves the subject P to be scanned and introduces the subject P into the inside of the rotating frame 17 of the gantry device 10. The bed device 30 includes, for example, a base 31, a bed driving device 32, a top board 33, and a support frame 34. The base 31 includes a housing that supports the support frame 34 in such a manner that the support frame 34 can move in the vertical direction (Y-axis direction). The bed driving device 32 includes a motor and an actuator. The bed driving device 32 moves the top board 33 along the support frame 34 in the longitudinal direction (Z-axis direction) of the top board 33. The bed driving device 32 also moves the top board 33 in the vertical direction (Y-axis direction). The top board 33 is a plate-shaped member on which the subject P is placed.

The bed driving device 32 may move not only the top board 33 but also the support frame 34 in the longitudinal direction of the top board 33. In contrast with the above, the gantry device 10 may be movable in the Z-axis direction, and the rotating frame 17 may be controlled to come around the subject P according to movement of the gantry device 10. Alternatively, both the gantry device 10 and the top board 33 may be movable. The X-ray CT apparatus 1 may be an apparatus in which the subject P is scanned in a standing or sitting position. In this case, the X-ray CT apparatus 1 has a subject support mechanism instead of the bed device 30, and the gantry device 10 rotates the rotating frame 17 around the axial direction perpendicular to the floor surface.

The console device 40 includes, for example, a memory 41, a display 42, an input interface 43, and processing circuitry 50. Although the first embodiment is described on the assumption that the console device 40 is separate from the gantry device 10, the gantry device 10 may include some or all of the components of the console device 40.

The memory 41 is realized, for example, by a semiconductor memory element such as a random access memory (RAM) or a flash memory, a hard disk, an optical disk, or the like. The memory 41 stores therein, for example, detection data, projection data, reconstructed image data, information about the subject P, imaging condition(s), and collection condition(s) of correction data. The memory 41 stores therein, for example, count data regarding a plurality of energy bins transmitted from the gantry device 10. Such data may be stored in an external memory with which the X-ray CT apparatus 1 can communicate, not in the memory 41 (or in addition to the memory 41). For example, the external memory is controlled by a cloud server that manages the external memory, the cloud server receiving a read/write request.

The display 42 displays various types of information. For example, the display 42 displays a reconstructed image (medical image, CT image) generated by the processing circuitry, a graphical user interface (GUI) image through which various operations by a user such as a doctor or technician are received, or other images. The display 42 is, for example, a liquid crystal display, a cathode ray tube (CRT), an organic electroluminescence (EL) display, or the like. The display 42 may be provided on the gantry device 10. The display 42 may be of a desktop type, or may be a display device (for example, a tablet terminal) capable of wireless communication with the main part of the console device 40. The display 42 is an example of a display.

The input interface 43 receives each of various input operations of the user and outputs an electrical signal indicating the content of the received input operation to the processing circuitry 50. The input interface 43 receives, for example, designation regarding a weighting factor by the user. The weighting factor includes a coefficient set for each of a plurality of pieces of count data (for example, a piece for the first count data and a piece for the second count data whose bin's energy bands are different) included in the detection data. As a designation regarding the weighting factor, the user may designate an energy band for adjusting the weighting factor or may designate the ratio of weighting factors between energy bands (the magnitude of the weighting factor in each energy band).

The input interface 43 is realized by, for example, a mouse, a keyboard, a touch panel, a trackball, a switch, a button, a joystick, a camera, an infrared sensor, a microphone, and/or the like. The input interface 43 may be provided in the gantry device 10. The input interface 43 may also be realized by a display device (for example, a tablet terminal) capable of wireless communication with the main part of the console device 40.

In the present specification, the input interface is not limited to one equipped with a physical operating part such as a mouse or a keyboard. For example, examples of the input interface also include a processing circuit handling an electrical signal, which receives an electrical signal corresponding to an input operation from an external input device provided separately from the apparatus and outputs this electrical signal to the control device.

The processing circuitry 50 controls the overall operation of the X-ray CT apparatus 1, the operation of the gantry device 10, the operation of the bed device 30, and a calibration operation for collecting correction data. The processing circuitry 50 includes as composing elements, for example, a system control function 51, a preprocessing function 52, the reconstruction function 53, an image processing function 54, the scan control function 55, and others.

These composing elements are realized by, for example, a hardware processor (computer) executing a program (software) stored in the memory 41. The hardware processor refers to circuitry such as a CPU, a graphics processing unit (GPU), an application specific integrated circuit (ASIC), a programmable logic device (for example, a simple programmable logic device (SPLD) or a complex programmable logic device (CPLD), or a field programmable gate array (FPGA)). Instead of storing the program in the memory 41, a configuration can be adopted in which the program is directly incorporated in a circuit of the hardware processor.

In this case, the hardware processor implements the functions by reading and executing the program incorporated in the circuit. The hardware processor is not limited to one configured as single circuitry, but may be configured as a single hardware processor by combining two or more independent circuits to implement each function. In another respect, two or more composing elements may be integrated into a single hardware processor to implement each function.

Composing elements of the console device 40 or the processing circuitry 50 may be distributed and realized by a plurality of pieces of hardware. The processing circuitry 50 may be realized by a processing device capable of communicating with the console device 40, not in a configuration in which the console device 40 has the processing circuitry 50. The processing device mentioned herein is, for example, a workstation connected to one X-ray CT apparatus, or a device (for example, a cloud server) that is connected to two or more X-ray CT apparatuses and collectively executes processes equivalent to those in the processing circuitry 50 which will be described below.

The system control function 51 controls various functions of the processing circuitry 50 based on input operations received by the input interface 43.

The preprocessing function 52 generates projection data by performing preprocessing on detection data including count data outputted by the DAS 16 and adjusted count data generated by an adjustment function 56. The preprocessing includes, for example, logarithmic transformation processing, offset correction processing, inter-channel sensitivity correction processing, beam hardening correction, scattered radiation correction, dark count correction, and the like.

The reconstruction function 53 generates reconstructed image data by performing reconstruction processing on the projection data generated by the preprocessing function 52, the reconstruction processing using a filtered back projection method, a successive approximation reconstruction method, or the like. The reconstruction function 53 stores the reconstructed image data obtained by the reconstruction in the memory 41. The reconstruction function 53 may perform reconstruction processing using the count data included in the detection data and the adjusted count data generated by the adjustment function 56 in a case where preprocessing is not performed by the preprocessing function 52, or other cases.

The reconstructed image data obtained by the reconstruction of the reconstruction function 53 may be, for example, two-dimensional image data for generating a cross-sectional image of an axial section perpendicular to the body axis of the subject P, and the like, two-dimensional image data for generating a cross-sectional image of another section, or three-dimensional image data. A generated three-dimensional image may have been subjected to rendering processing. When generating the three-dimensional image data, the reconstruction function 53 may transform projection data for generating a two-dimensional image or reconstructed image data into three-dimensional image data. Transformation to three-dimensional image data may be performed by the preprocessing function 52.

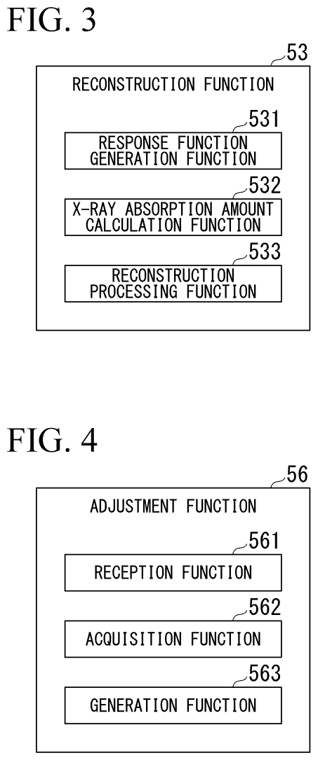

FIG. 3 is a diagram showing an example of functional blocks of the reconstruction function 53 according to the embodiment. The reconstruction function 53 includes, for example, a response function generation function 531, an X-ray absorption amount calculation function 532, and a reconstruction processing function 533. The response function generation function 531 generates data of a response function that represents detector response characteristics. For example, the response function generation function 531 measures a response (i.e., detected energy and detected intensity) of a standard detection system to a plurality of monochromatic X-rays having a plurality of incident X-ray energies on the basis of predictive calculation, experiment, and a combination of predictive calculation and experiment, and generates a response function based on the measured values of the detected energy and detected intensity. The response function generation function 531 may also generate response function data based on measured values obtained by actual measurement that have been collected during calibration or the like. The response function specifies the relationship between the detected energy and the output response of the system for each incident X-ray. For example, the response function specifies the relationship between the detected energy and the detected intensity for each incident X-ray. Data of the generated response function is stored in the memory 41.

The X-ray absorption amount calculation function 532 calculates the X-ray absorption amount for each of a number of basis materials on the basis of projection data, adjusted count data generated by the adjustment function 56, the energy spectrum of X-rays incident on the subject P, and the response function stored in the memory 41. The X-ray absorption amount calculation function 532 can calculate the X-ray absorption amount that is not affected by the response characteristics of the X-ray detector 15 and the DAS 16 since the X-ray absorption amount calculation function 532 calculates the X-ray absorption amount on the basis of the adjusted count data and the energy spectrum of the X-rays incident on the subject P using the response function. Processing of obtaining the X-ray absorption amount for each basis material is also called material discrimination.

As basis materials, any materials can be set, such as calcium, calcific substance, bone, fat, muscle, air, organ, lesion, hard tissue, soft tissue, contrast material, etc. When a number of types of contrast materials are used as basis materials, each contrast material can be set as a basis material. The type of a basis material to be calculated may be determined in advance by a user or the like via the input interface 43. A contrast material includes at least one of gadolinium, gold, and bismuth, for example. The contrast material does not have to include these materials. The X-ray absorption amount indicates the amount of X-rays absorbed by a basis material. For example, the X-ray absorption amount is defined by a combination of an X-ray attenuation coefficient and an X-ray transmission path length.

The reconstruction processing function 533 generates reconstructed image data for displaying a reconstructed image expressing the spatial distribution of a basis material to be visualized among the plurality of basis materials on the basis of the X-ray absorption amount for each of the basis materials calculated by the X-ray absorption amount calculation function 532, and stores the generated reconstructed image data in the memory 41. The basis material to be visualized may be one type of basis material or two or more types of basis materials. The type of the basis material to be visualized may be determined by a user or the like via the input interface 43.

Referring back to FIG. 1, the image processing function 54 generates a reconstructed image based on the reconstructed image data generated by the reconstruction processing function 533. The image processing function 54 causes the display 42 to display the generated reconstructed image. The image processing function 54 causes the display 42 to display, as the reconstructed image, an image of a body trunk of the subject P and/or an image of a contrast agent injected into the subject P, for example. The image processing function 54 is an example of an image processor.

The scan control function 55 controls detection data collection processing in the gantry device 10 by issuing instructions to the X-ray high voltage device 14, the DAS 16, the control device 18, the gantry driving device 19, and the bed driving device 32. The scan control function 55 controls the operation of each part when capturing images to collect positioning images and capturing images to be used for diagnosis.

FIG. 4 is a diagram showing an example of function blocks of the adjustment function 56 according to the embodiment. The adjustment function 56 includes, for example, a reception function 561, an acquisition function 562, and a generation function 563. The adjustment function 56 generates adjusted count data obtained by weighting a plurality of pieces of count data included in detection data.

The reception function 561 receives designation regarding a weighting factor, for example, designation of a weighting factor for each of a number of energy bands. The designation regarding a weighting factor is transmitted, for example, through the input interface 43 or the operation console 80 operated by the user. The reception function 561 is an example of a receiver.

The acquisition function 562 acquires count data of X-ray photons included in the detection data obtained by detection of the X-ray detector 15. The count data of X-ray photons includes four pieces/kinds of count data having their respective different energy bands, which are, for example, first count data, second count data, third count data, and fourth count data. The acquisition function 562 acquires the first count data to the fourth count data whose energy bands are different from each other. The acquisition function 562 is an example of an acquirer.

The generation function 563 sets first to fourth weighting factors to the first count data to the fourth count data acquired by the acquisition function 562, respectively. The first to the fourth weighting factors are weighting factors relative to one another. The generation function 563 generates adjusted count data based on the first count data to the fourth count data and the first to the fourth weighting factors. The generation function 563 is an example of a generator.

The operation console 80 includes, for example, an input device, a support stand, and a display device. The input device is realized by, for example, a mouse, a keyboard, a touch panel, a trackball, a switch, a button, a joystick, a camera, an infrared sensor, a microphone, and/or the like similarly to the input interface 43 in the console device 40.

The support stand is placed on the floor surface, for example, and supports the input device and the display device. Casters are attached to a bottom surface of the support stand in such a manner that the support stand can be moved within the examination room or the operation room. The support stand may be installed in the examination room or the operation room in a placed or fixed state. The operation console 80 may be attached to the gantry device 10 or the bed device 30.

The display device is realized by, for example, a display unit or a touch panel. If the display device is a touch panel, the display device also functions as an input device. The display device displays various types of information. The operation console 80, similarly to the input interface 43, receives various input operations of the user and outputs electrical signals indicating the contents of the received input operations to the processing circuitry 50. The operation console 80 receives designation regarding a weighting factor from the user.

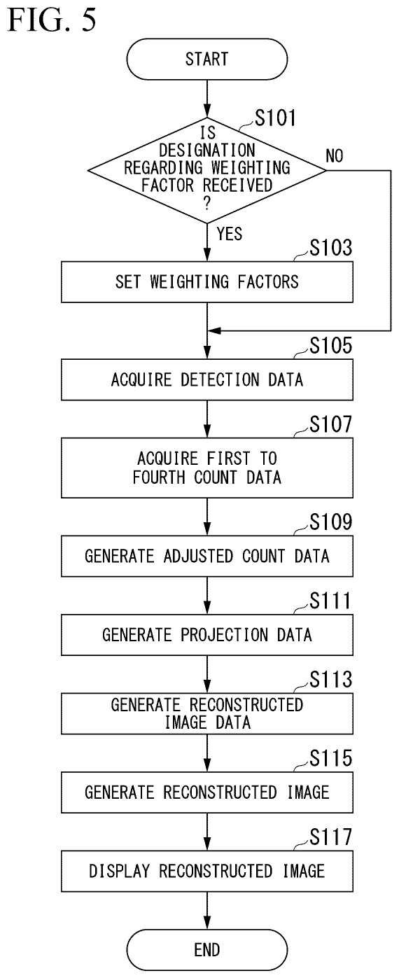

Next, processing in the X-ray CT apparatus 1 will be described. FIG. 5 is a flowchart showing an example of processing in the X-ray CT apparatus 1. A process of the flowchart shown in FIG. 5 is executed for each view after the X-ray CT apparatus 1 starts examining the subject P, in other words, in real time.

When examination of the subject starts, if the user intends to designate a weighting factor, the user performs an input operation on the input interface 43 or the operation console 80 in order to designate the weighting factor. The input interface 43 or the operation console 80 on which the input operation has been performed transmits an electrical signal indicating information regarding the weighting factor corresponding to the input operation to the processing circuitry 50.

For example, the user designates a weighting factor for each of the first, second, third, and fourth count data as information on weighting factors. The user may designate the weighting factor or factors for one or some of the first count data to the fourth count data and keep the weighting factor or factors for the remaining count data at a value or values that have been set in advance.

The information on weighting factors is numerical information designated by the user who performs examination and the like by his or her own input operation on the input interface 43 or the operation console 80. The user designates weighting factors according to the content of an image to be captured by the X-ray CT apparatus 1 in accordance with the content of the examination. For example, when the user wants to improve the visibility of a trunk of a living body and view the trunk in more detail, the user designates a weighting factor that increases weighting for count data in a bin whose energy band is high. For example, when the user wants to improve the visibility of a contrast agent injected into the subject P and view the contrast agent in more detail, the user designates a weighting factor that increases weighting for count data in a bin whose energy band is low.

The reception function 561 included in the adjustment function 56 in the processing circuitry 50 determines whether or not information on weighting factors transmitted by the input interface 43 or the console 80, for example, information of the weighting factor, has been received (step S101). If it is determined that the information of the weighting factor has been received, the generation function 563 sets a weighting factor for count data of a bin for each energy band (step S103). If it is determined that the information of the weighting factor has not been received, the generation function 563 skips the processing of step S103 and proceeds to step S105.

In the gantry device 10, the DAS 16 generates detection data based on count data obtained by the X-ray detector 15 counting X-ray photons contained in X-rays radiated by the X-ray tube 11 for each energy band, and outputs the detection data to the processing circuitry 50. In the processing circuitry 50, the acquisition function 562 included in the adjustment function 56 acquires the detection data outputted (step S105).

Subsequently, the acquisition function 562 extracts and acquires the first to fourth count data pieces from the acquired detection data (step S107). Subsequently, the generation function 563 generates adjusted count data based on the first count data to the fourth count data and the first weighting factor to the fourth weighting factor (step S109).

The generation function 563 multiplies the first count data to the fourth count data by the first weighting factor to the fourth weighting factor, respectively, and calculates the sum of the multiplication results, for example. The generation function 563 divides the first count data to the fourth count data by the calculated sum to thereby generate adjusted count data including pieces of the first to fourth count data which have been adjusted (hereinafter, first adjusted count data to fourth adjusted count data).

Subsequently, the preprocessing function 52 generates projection data using the adjusted count data generated by the adjustment function 56 (step S111). If preprocessing is not performed, the processing of step S111 is skipped. Then, the reconstruction function 53 performs reconstruction processing on the projection data generated by the preprocessing function 52 to generate reconstructed image data (step S113). Since the projection data is data generated using the adjusted count data, the reconstructed image data generated based on the projection image also amounts to data generated using the adjusted count data.

Subsequently, the image processing function 54 generates a reconstructed image based on the reconstructed image data generated by the reconstruction function 53 (step S115). Then, the image processing function 54 causes the display 42 to display the generated reconstructed image (step S117). After the steps mentioned above, the X-ray CT apparatus 1 ends processing performed during one view.

In the X-ray CT apparatus 1 of the first embodiment, a weighting factor is set for each of a number of count data pieces for each energy band included in the detection data to perform weighting and subject the count data to reconstruction. Accordingly, it is possible to adjust the amount of processing for each energy band at the time of generating the reconstructed image data.

Therefore, by increasing the weighting of count data for an energy band considered to be necessary for an image by the user or the like and decreasing the weighting of count data for the other energy bands or band, it is possible to reduce the amount of processing at the time of generating an entire image while generating a clear image for the energy band considered to be necessary for the image by the user or the like. This makes it possible to generate, in a short time, a reconstructed image in which a part that the user or the like needs is clearly displayed.

In the first embodiment described above, the generation function 563 generates adjusted count data by weighting the count data with a weighting factor set theretofore the count data, but the generation function 563 may perform weighting all at once, or may perform weighting while gradually varying the weighting factor in stages or step-by-step in accordance with the amount of variation in the weighting factor for the count data. Such gradual variation in the weighting factor may be performed within a defined period of time, for example, within a period of time over which several views have passed. A time for several views to elapse is an example of a first period.

For example, when the user designates a weighting factor, weighting factor setting may be realized in such a manner that the designated weighting factor is set while one view is being executed. Alternatively, the weighting factor may be varied in stages while several views are being executed, and the designated weighting factor may be set after the several views have been executed.

The manner in which weighting is varied in stages, mentioned above, may be a nonlinear variation such as a stepwise variation, or a linear variation. In the following description, a weighting factor set for count data is sometimes referred to as a weighting factor set to a bin for collecting the count data.

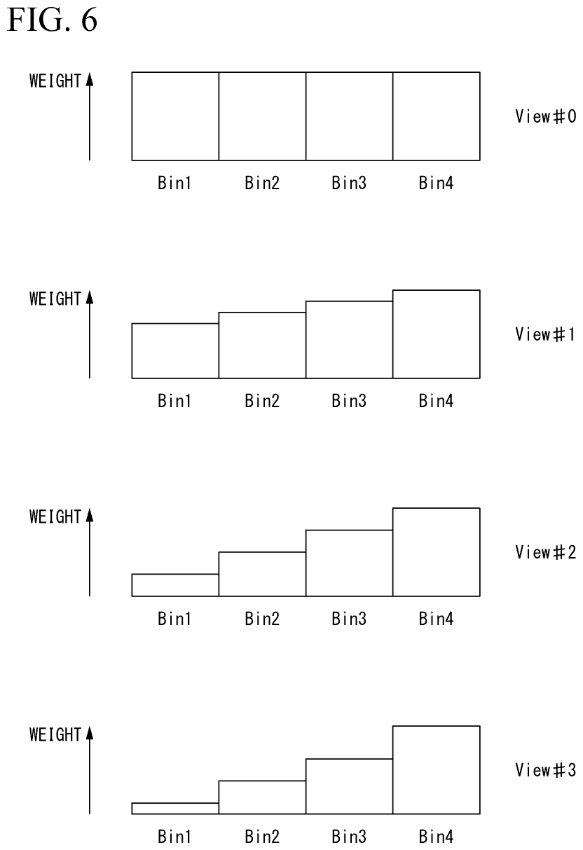

FIG. 6 is a diagram visualizing changes in the ratio of weighting factors each time one view is executed. In this example, there is described an example of setting a weighting factor designated by the user as a weighting factor in weighting the count data of each bin after three views have been executed. Here, X-ray photons in the lowest energy band are collected in a first bin Bin1, and X-ray photons in higher energy bands are collected in a second bin Bin2, a third bin Bin3, and a fourth bin Bin4 in this order. X-ray photons in the highest energy band are collected in the fourth bin Bin4.

First, in a 0-th view (View #0) in a state in which designation of a weighting factor has not yet been performed, the generation function 563 has not set weighting factors among the first bin Bin1 to the fourth bin Bin4. Therefore, the weighting factors of the first bin Bin1 to the fourth bin Bin4 are weighting factors (all 1) that have been set in advance, and are mutually constant.

Here, for example, in order to make the image of the body trunk clearer, the user sets the weighting factors, and the weight of the fourth bin Bin4 is set large. In this case, the generation function 563 first adjusts the weighting factors of the first bin Bin1 to the third bin Bin3 in the first view (View #1), respectively, to values that are not as low as the weighting factors designated by the user.

Subsequently, the generation function 563 adjusts the weighting factors of the first bin Bin1 to the third bin Bin3 in the second view (View #2), respectively, to values that are lower than the weighting factors of the first bin Bin1 to the third bin Bin3 set in the first view (View #1) and that are not as low as the weighting factors designated by the user. Then, the generation function 563 sets the weighting factors of the first bin Bin1 to the third bin Bin3 in the third view (View #3) to the weighting factors designated by the user.

In this way, when weighting the count data, the weighting factors may be changed in stages over time and weighting may be performed in stages. In this case, it is possible to prevent the weighting factors from changing suddenly. Therefore, it is possible to prevent a reconstructed image generated from changing suddenly.

In addition, in the first embodiment described above, the user designates weighting factors as information on weighting factors, but instead of the weighting factors, a target of interest (hereinafter, target of interest) in the subject P may be designated, and weighting factors for generating an image in which the designated target is clearer may be automatically set. The target of interest may be, for example, a body trunk of the subject P, or a contrast agent injected into the subject P.

The memory 41 stores therein, for example, a weighting factor setting table indicating a target of interest and a combination of a plurality of pieces of count data and weighting factors preset in association with the target of interest. The user designates a target of interest by performing an input operation on the input interface 43 or the operation console 80, for example. The input interface 43 or the operation console 80 outputs an electrical signal indicating the designated target of interest to the processing circuitry 50. A combination of a plurality of pieces of count data having been preset and weighting factors may be a combination other than the combination corresponding to the target of interest.

Upon acquiring the electrical signal indicating the target of interest, the processing circuitry 50 reads out the weighting factor setting table from the memory 41. The generation function 563 in the processing circuitry 50 uses the acquired electrical signal and the read-out weighting factor setting table to identify and set the weighting factors associated with the target of interest.

In this way, the weighting factor may be set by the user designating a target of interest instead of the weighting factor. The weighting factor associated with the target of interest may be associated with some or all of the plurality of pieces of count data. The weighting factor associated with the target of interest may be a fixed value, or may be associated with a numerical value according to change over time. In this case, the weighting factor may be associated with it for each view, for example.

In the first embodiment described above, when the user designates a weighting factor, the weighting factor is adjusted manually, but the weighting factor may be adjusted in other ways, for example, automatically. For example, an SD value of each piece of data such as projection data or reconstructed image data may be calculated, and a weighting factor may be adjusted based on the calculated SD value. For example, the weighting factor may be adjusted when the calculated SD value exceeds a predetermined threshold value.

### Second Embodiment

Next, a second embodiment will be described. An X-ray CT apparatus 1 of the second embodiment has a different procedure for generating a reconstructed image as compared to the first embodiment. The X-ray CT apparatus 1 of the second embodiment generates a reconstructed image by using reconstructed image data in which weighting factors of the count data have not been adjusted and reconstructed image data in which weighting factors of the count data have been adjusted. The X-ray CT apparatus 1 of the second embodiment will be described below, focusing on differences from the first embodiment. In the following description, components and functions common to the first embodiment may be omitted with the same reference symbols as in the first embodiment being assigned to them.

In the X-ray CT apparatus 1 of the second embodiment, the adjustment function 56 generates adjusted count data in the same procedure as in the first embodiment. The preprocessing function 52 generates first projection data based on count data (hereinafter, unadjusted count data) included in detection data outputted by the DAS 16, and generates second projection data based on the adjusted count data generated by the adjustment function 56.

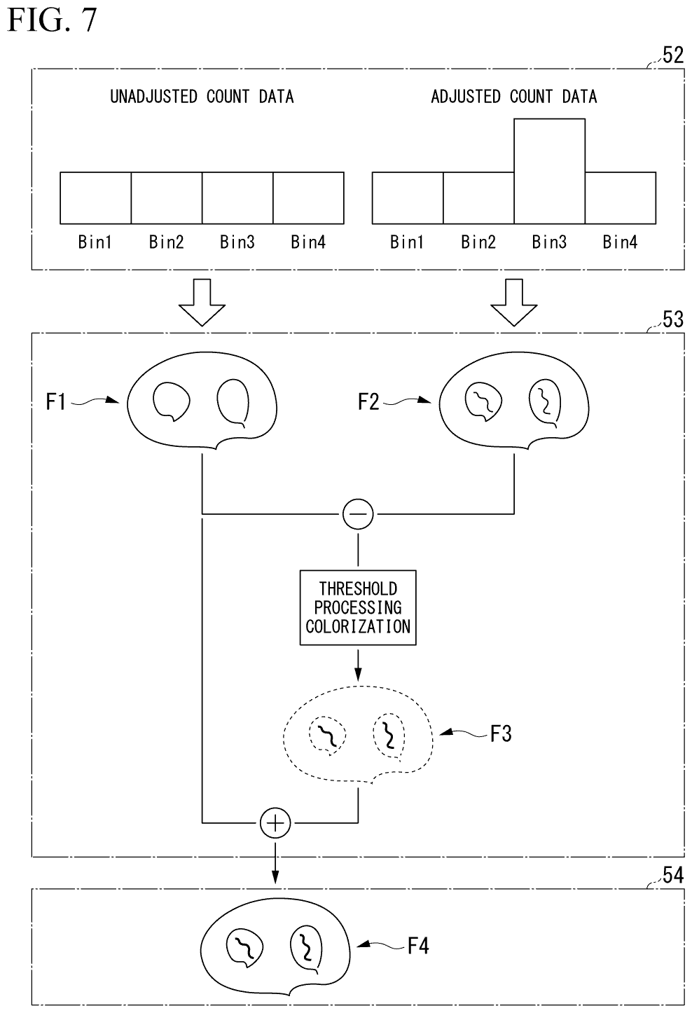

The reconstruction function 53 generates reconstructed image data using the first projection data and the second projection data that are generated by the preprocessing function 52, and the image processing function 54 generates a reconstructed image based on the reconstructed image data generated by the reconstruction function 53 and causes the display 42 to display the reconstructed image. Hereinafter, the procedure for generating a reconstructed image will be described with reference to FIG. 7.

FIG. 7 is a diagram illustrating an example of a process of generating a reconstructed image. In the X-ray CT apparatus 1 of the second embodiment, the preprocessing function 52 acquires unadjusted count data included in the detection data and adjusted count data generated by the adjustment function 56. The unadjusted count data is, for example, count data in which weighting of each energy band is not adjusted and is constant with no change.

On the other hand, the adjusted count data is count data obtained by performing weighting designated by the user on the raw count data for each energy band. The adjusted count data in this example is count data in which the count data of the third bin has been weighted more heavily than the count data of the other bins. For example, shown is a premise that the energy band of the third bin is suitable for displaying a particular target of interest in the subject P.

The preprocessing function 52 generates first projection data based on the unadjusted count data and second projection data based on the adjusted count data. The reconstruction function 53 generates first reconstructed image data and second reconstructed image data on the basis of the first projection data and the second projection data, respectively. The first reconstructed image data is an example of unadjusted reconstructed image data.

A first reconstructed image Flin which the first reconstructed image data is visualized and a second reconstructed image F2 in which the second reconstructed image data is visualized are both shown in FIG. 7. The first reconstructed image F1 is, for example, an image that uniformly represents a specific region of the subject P, and the second reconstructed image F2 is, for example, an image that represents an image of a target of interest in a more prominent state than images of arears other than the target. The first reconstructed image F1 is an example of an unadjusted reconstructed image.

Subsequently, the reconstruction function 53 subtracts the unadjusted count data from the adjusted count data to obtain a difference therebetween. The reconstruction function 53 performs threshold processing and image colorization on difference data obtained as the difference between the adjusted count data and the unadjusted count data to generate emphasized count data.

In FIG. 7, there is shown an emphasized reconstructed image F3 in which the emphasized count data is visualized. The emphasized reconstructed image F3 is, for example, an image obtained by extracting the target of interest from the second reconstructed image F2 generated based on the adjusted count data. Solid line portions included in the emphasized reconstructed image F3 in FIG. 7 are portions remaining after the target of interest is extracted from the emphasized reconstructed image F3.

The image processing function 54 generates the first reconstructed image F1 and the emphasized reconstructed image F3 based on the first reconstructed image data and the emphasized reconstructed image data that are generated by the reconstruction function 53. The image processing function 54 generates a superimposed reconstructed image F4 by superimposing the first reconstructed image on the emphasized reconstructed image or vice versa. The image processing function 54 causes the display 42 to display the generated superimposed reconstructed image F4.

The X-ray CT apparatus 1 of the second embodiment has substantially the same effects as the X-ray CT 1 of the first embodiment. In addition to the effects, the X-ray CT apparatus 1 of the second embodiment performs threshold processing and image colorization on the difference data obtained as the difference between the adjusted count data and the unadjusted count data to generate the emphasized count data. By doing so, in the X-ray CT apparatus 1 of the second embodiment, it is possible to show the user the superimposed reconstructed image F4 that is equivalent to the first reconstructed image F1 in which the target of interest generated based on the count data has been emphasized.

### Third Embodiment

Next, a third embodiment will be described. The third embodiment differs from the first embodiment in that the processing circuitry 50 in the console device 40 does not have the adjustment function 56, and the control device 18 has the adjustment function. The third embodiment will be described below, focusing on differences from the first embodiment.

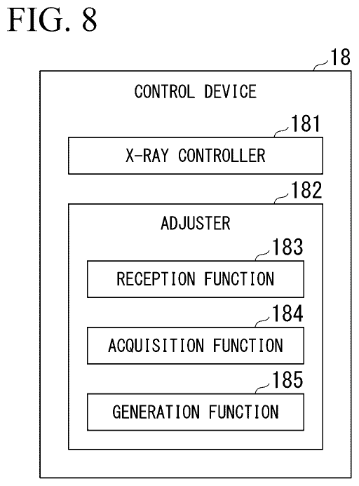

FIG. 8 is a diagram showing an example of the control device 18 of the third embodiment. In the third embodiment, the control device 18 includes, for example, an X-ray controller 181 and an adjuster 182. The X-ray controller 181 has functions substantially equal to that of the control device 18 in the first embodiment. The adjuster 182 includes, for example, a reception function 183, an acquisition function 184, and a generation function 185.

The reception function 183 receives designation regarding a weighting factor, for example, designation of a weighting factor for each of a number of energy bands. The designation regarding a weighting factor is transmitted, for example, by the operation console 80 operated by the user or the input interface 43 in the console device 40.

The acquisition function 184 acquires count data of X-ray photons included in detection data obtained by detection of the X-ray detector 15, similarly to the acquisition function 562 in the first embodiment. The generation function 185 sets first to fourth weighting factors for the first count data piece to the fourth count data piece acquired by the acquisition function 184, respectively, similarly to the generation function 563 in the first embodiment, and generates adjusted count data based on these count data pieces and weighting factors. The generation function 185 changes the generated adjusted count data to include the detection data and outputs the changed data to the console device 40.

The X-ray CT apparatus 1 of the third embodiment has substantially the same effects as the X-ray CT apparatus 1 of the first embodiment. Furthermore, as in the X-ray CT apparatus 1 of the third embodiment, by setting weighting factors for count data in the control device 18, the processing load in the console device 40 can be distributed to the control device 18, and thus a reconstructed image can be generated in a short time accordingly.

In addition, in each of the above embodiments, the generation function 563 may determine whether or not a metal is contained within the detection range of the X-ray detector 15 in the body of the subject P, and if it is determined that a metal is contained, the generation function 563 may increase the weight for count data of a high energy band. If it is determined that a metal is contained within the subject P, metal artifacts in a reconstructed image can be suppressed by increasing the weight for the count data of the high energy band.

According to at least one of the above-described embodiments, it is possible to generate a reconstructed image in a short time by a configuration including: an acquirer that acquires at least first count data and second count data having different energy bands as count data of X-ray photons detected by an X-ray detector; a generator that generates adjusted count data based on relative weighting factors between the first count data and the second count data; and a reconstructor that reconstructs the adjusted count data.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray CT apparatus comprising processing circuitry configured to:
acquire at least first count data and second count data having different energy bands as count data of X-ray photons detected by an X-ray detector;
generate adjusted count data based on relative weighting factors between the first count data and the second count data; and
reconstruct the adjusted count data.

2. The X-ray CT apparatus according to claim 1, wherein the processing circuitry is configured to generate the adjusted count data in real time.

3. The X-ray CT apparatus according to claim 1, wherein the processing circuitry is configured to vary the weighting factors in stages according to an amount of variation of the weighting factors of the count data.

4. The X-ray CT apparatus device according to claim 3, wherein the processing circuitry is configured to vary the weighting factors during a first period.

5. The X-ray CT apparatus according to claim 1, wherein the processing circuitry is further configured to receive designation regarding the weighting factors.

6. The X-ray CT apparatus according to claim 5, wherein the processing circuitry is configured to receive designation of the weighting factors.

7. The X-ray CT apparatus according to claim 5, wherein the processing circuitry is configured to receive designation for an energy band for which a weighting factor is to be increased or decreased.

8. The X-ray CT apparatus according to claim 1, wherein the processing circuitry is configured to generate the adjusted count data based on a target of interest in a subject.

9. The X-ray CT apparatus according to claim 8, wherein the target of interest includes a trunk of the subject.

10. The X-ray CT apparatus according to claim 8, wherein the target of interest includes a contrast agent injected into the subject.

11. The X-ray CT apparatus according to claim 1, wherein the processing circuitry is configured to generate the adjusted count data based on a preset combination of the weighting factors for the first count data and the second count data.

12. The X-ray CT apparatus according to claim 5, wherein the processing circuitry is configured to receive designation regarding the weighting factors transmitted by an operation console operated by a user.

13. The X-ray CT apparatus according to claim 1, wherein the processing circuitry is configured to generate reconstructed image data based on the count data and the adjusted count data.

14. The X-ray CT apparatus according to claim 13, wherein the processing circuitry is configured to:
cause a display to display a reconstructed image based on reconstructed image data obtained by reconstructing the adjusted count data;
generate emphasized count data based on difference data obtained by subtracting the count data from the adjusted count data; and
cause the display to display a superimposed reconstructed image obtained by superimposing an emphasized reconstructed image obtained by reconstructing the emphasized count data on an unadjusted reconstructed image based on unadjusted reconstructed image data obtained by reconstructing the count data.

15. The X-ray CT apparatus according to claim 1, comprising:
a control device configured to control the X-ray detector;
a gantry device having the X-ray detector and the control device built into the gantry device; and
a console device provided outside the gantry device,
wherein an acquisition function and a generation function of the processing circuitry are provided in the control device and a reconstruction function of the processing circuitry is provided in the console device, and
wherein the adjusted count data is generated by the control device, and the generated adjusted count data is transmitted to the console device.

16. The X-ray CT apparatus according to claim 5, wherein the processing circuitry is configured to increase a weight of count data in a high energy band when it is determined that a metal is contained within a detection range of the X-ray detector in a body of the subject.

17. A method of controlling an X-ray CT apparatus, using a computer, comprising:
acquiring at least first count data and second count data having different energy bands as count data of X-ray photons detected by an X-ray detector;
generating adjusted count data based on relative weighting factors between the first count data and the second count data; and
reconstructing the adjusted count data.

18. A computer-readable non-transitory storage medium storing a program causing a computer to:
acquire at least first count data and second count data having different energy bands as count data of X-ray photons detected by an X-ray detector;
generate adjusted count data based on relative weighting factors between the first count data and the second count data; and
reconstruct the adjusted count data.
